(19) United States
(12) Patent Application Publication
(10) Pub. No.: US 2005/0197512 A1
(43) Pub. Date: Sep. 8, 2005

(54) Purification process

(76) Inventors: Ulrich Beutler, Oberwil (CH); Peter Funfschilling, Allschwil (CH); Gerhard Penn, Oberwil (CH); Alfons Roth, Heitersheim (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/075,227

(22) Filed: Mar. 8, 2005

Related U.S. Application Data
(63) Continuation-in-part of application No. 10/522,828.

(30) Foreign Application Priority Data
Aug. 29, 2003 (GB) 0320312.2
Feb. 25, 2005 (GB) 0503942.5

(51) Int. Cl. C07C 29/86

(52) U.S. Cl. 564/384

(57) ABSTRACT

Process for the preparation of pure allylamine pharmaceutical terbinafine of formula I 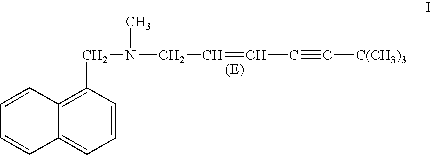 in free base form or acid addition salt form, by distilling crude terbinafine base, preferably by short path distillation, e.g. at a temperature above 100° C. and reduced pressure, e.g. 0.2 mbar, and recovering the purified product in free base or acid addition salt form. The process is indicated for use in removal of metal or non-metal contaminants, optionally together with salt formation under simultaneous precipitation of pure trans isomer.
Detection of non-metal contaminants such as substance A of formula 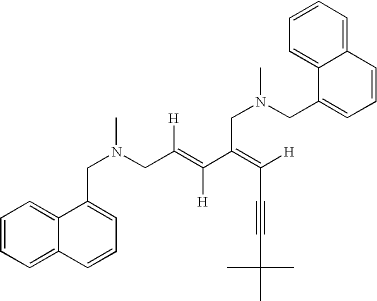 preferably is effected by RP HPLC analysis with UV detection.

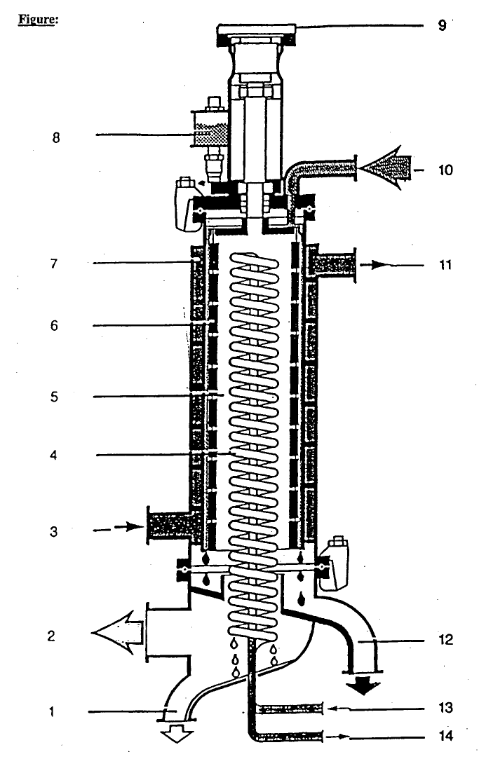

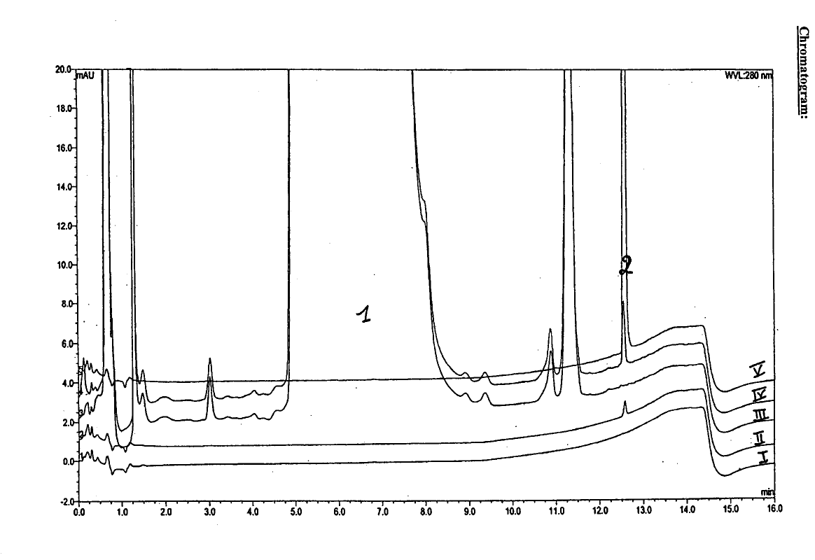

The invention relates to a purification process for an allylamine pharmaceutical. It concerns a process for purifying crude terbinafine base.

Terbinafine, particularly in the form of the hydrochloride acid addition salt form, is known from e.g. EP 24587. It belongs to the class of allylamine antimycotics. It is commercially available under the trademark Lamisil®. It is effective upon both topical and oral administration, in a wide range of fungal infections. Terbinafine is particularly useful against dermatophytes, contagious fungi that invade dead tissues of the skin or its appendages such as stratum corneum, nail, and hair.

Terbinafine represents a significant advance in antifungal therapy based on its potent fungicidal action in vitro and rapid clinical efficacy in various dermatophyte infections when given orally as well as topically. It is a potent inhibitor of ergosterol biosynthesis (Ann. NY Acad. Sci. 544 [1988] 46-62), it blocks the action of squalene epoxidase, thus inhibiting the transformation of squalene to squalene epoxide. Although ergosterol synthesis is only partially inhibited, cell growth is completely arrested. This suggests that the fungicidal effect of terbinafine may be related to the accumulation of squalene, which at high concentrations may be toxic to the fungus. The spectrum of activity of terbinafine in vitro embraces all dermatophytes of the genera Trichophyton, Epidermophyton and Microsporum. The mean minimum inhibitory concentrations for these dermatophytes range from 0.001 μg/ml to 0.01 μg/ml (Science 224 [1984] 1239-1241). Terbinafine is also active in vitro against molds and dimorphic fungi, and against many pathogenic yeasts of the genera Pityrosporum, Candida and Rhodotorula.

The structure of terbinafine is as shown in formula I 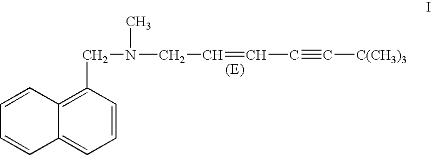
and its chemical name is i.a. (E)-N-(6,6-dimethyl-2-hepten-4-ynyl)-N-methyl-1-naphthalene methanamine.

It may be in free base form or in acid addition salt form. An acid addition salt form can be prepared from the free base form in conventional manner and vice-versa. Examples of suitable acid addition salt forms are the hydrochloride, the lactate, the ascorbate and the malate, e.g. the L-(−)-hydrogenmalate. The free base and the hydrochloride and malate salts are preferred, especially the hydrochloride and the L-(−)-hydrogenmalate.

As appears from formula I above, terbinafine is an allylamine compound with a triple bond conjugated with a double bond in the side chain. Terbinafine was invented many years ago (see e.g. EP 24587, Example 16), and such conjugated enyne structure was, and still is, highly unusual in the pharmaceutical field, constituting a novel structural feature in medicinal chemistry.

Both double and triple bonds are usually highly reactive. While the chemical literature does not exclude that compounds with such structure may be stable, some are unstable and may decompose upon storage or processing, such as when heat is applied, as e.g. upon distillation at elevated temperatures.

Thus it appears from e.g. E. R. H. Jones et al., J. Chem. Soc. (1960) 341-346 that submitting pure penta-1,2-dien-4-yne to simple distillation at its normal boiling temperature of 57°, already results in decomposition. Similarly, the (non-conjugated) 1-alken-4-yne dimer [CH₂═CH—CH₂—C≡C—C(CH₃)(OH)—]₂, i.e. 6,7-dimethyl-dodeca-1,11-dien-4,8-diyn-6,7-diol (compound V in H. Disselnkötter and P. Kurtz, Ann. Chem. [1964] 26-34) undergoes considerable decomposition upon distillation under reduced temperature (85-90° C.) and pressure (0.05 mm Hg), as well as upon renewed distillation at 81-85° C. and 0.03 mm Hg. Further, the enediyne (Z,Z)-3,7-decadiene-1,5,9-triyne polymerizes readily, and solutions thereof thermolyse at 170-190° C. to give naphthalene, while the thermolysis of the corresponding (E,Z) and (E,E) isomers gives other products or a polymer (J. Am. Chem. Soc. 114 [1992] 3120-3121).

Further, isomerization of conjugated enyne compounds, e.g. the ether CH₃CH═CH—C≡C—CH₂OC₂H₅ to the corresponding 1,3,5-triene compound may be accompanied by considerable polymeric residue after distillation, resulting from accompanying 1,6-elimination of ethanol, while replacement of the —OC₂H₅ group with an amino group results in aromatization (Van-Dongen, J. et al., Recueil Trav. Chim. Pays-Bas 86 [1967] 1077-1081).

Additionally, overall, remarkably, it appears from e.g. the above publications that when distillation is effected at all with enyne derivatives, this is usually effected at temperatures below or slightly above 100° C., especially below about 125° C., as is to be expected with highly reactive compounds susceptible of decomposition or degradation or polymerisation, or even explosion, upon heating. This appears also for most of the alkenyne derivatives disclosed in e.g. Recueil Trav. Chim. Pays-Bas 85 (1966) 952-965 and Zh. Org. Khim 3 (1967) 1792-3 (CA 68 [1968] 12370), while the two intermediates for pheromones disclosed in Czech Author's Certificate No. 232843 (CA 106 [1984] 213632b) are purified by distillation under reduced pressure at 102-115° C. and 118-125° C., respectively.

Further, terbinafine in free base form is boiling at 140° C. at 0.3 mbar pressure, and at that temperature its thermal stability is limited: thus the following decomposition can be observed (upon analysis by gas chromatography; the area under the peak of one compound relative to the sum of all peaks is named area-%; in the case of the Z-isomer, area-% should be approximately identical with weight-%):

| Heating time | By-product 1 | Z-isomer | Unchanged E-isomer |
|---|---|---|---|
| (h) | (area-%) | (area-%) | (area-%) |
| 0 | 0.09 | 0.25 | 97.6 |
| 7 | 0.57 | 0.34 | 96.6 |
| 23 | 0.92 | 0.45 | 94.7 |
| 32 | 1.20 | 0.52 | 92.0 |

By-product 1 = (methyl)(naphthalen-1-ylmethyl)amine

On the other hand the product solidifies already below 43° C.

One would therefore normally refrain from effecting operations requiring substantial application of heat when working-up a chemical compound with such an unusual structure, particularly when this is associated with limited thermal stability, especially in large-scale operations, such as in the industrial production of a pharmaceutical. For example, in Example 13 of Banyu EP 0 421302 A2 describing a preparation of terbinafine, the crude mixture (free base) obtained after reaction is subjected to purification by silica gel chromatography.

However, it has been found that, surprisingly and counter-intuitively, terbinafine base may be submitted to distillation with no particularly unfavourable effect. Further, it has been found that such distillation may be effected at elevated temperature, e.g. even at a temperature significantly higher than 100° C., e.g. from about 110° C. to about 170° C., preferably from about 125° C. to about 165° C., especially about 160° C., and under correspondingly reduced pressure, e.g. 0.2 mbar at 160° C. (jacket temperature).

The yield attained thereby is normally about 95% starting from crude product.

Further, it has also been found that such distillation may even be effected using large amounts of crude terbinafine base, i.e. in an industrial setting, e.g. in the large-scale production of purified terbinafine base and acid addition salts, e.g. in amounts of at least about 5 kg, preferably at least about 50 kg, especially at least about 200 kg, e.g. from about 500 kg to about 2 tons, more preferably from about 600 kg to about 900 kg, most preferably from about 800 kg to about 900 kg, especially about 850 kg purified product in free base form per distillation batch or run.

The invention therefore concerns a novel process for the purification of terbinafine comprising subjecting crude terbinafine in free base form to distillation and recovering the resultant product in free base or acid addition salt form, hereinafter briefly named “the process of the invention”.

The process of the invention is particularly useful for separating terbinafine from contaminants, e.g. metal contaminants resulting from its chemical synthesis, e.g. from catalysts, such as copper and/or, in particular, palladium contaminants, particularly for reducing or eliminating contaminants resulting from synthesis in accordance with or similarly to the processes described in e.g. Banyu EP 421302 and/or Dipharma EP 1'236'709, e.g. by reaction of (E)-N-(3-halo-2-propenyl)-N-methyl-N-(1-naphthylmethyl)amine (compound of formula IV of EP 421302 wherein R¹¹ is methyl, R²¹ is 1-naphthylmethyl and W is halogen, e.g. bromo, preferably chloro), with 3,3-dimethyl-1-butyne (compound of formula V thereof, wherein R⁷ is tert-butyl) in the presence of a palladium and/or a copper catalyst to obtain terbinafine base. The catalyst is e.g. copper(I)iodide, or copper(I)iodide together with bis-(triphenylphosphine)palladium-(II)-dichloride or tetrakis(triphenylphosphine)palladium, or a further palladium-, copper- or palladium/copper-containing catalyst selected from those disclosed in EP 421302 A2, e.g. on page 7, line 54 to page 8, line 18.

The process of the invention may be effected by conventional means. It preferably is effected as a so-called “gentle” distillation process. It may e.g. be effected as a batch distillation, or preferably in continuous or semi-continuous manner, and especially as a “short path” distillation, whereby the path between heating mantle and condensor is short, e.g. of the order of 10 cm, thus minimizing the time during which terbinafine is at an elevated temperature, e.g. above 100° C.

The term “short path distillation” is to be understood herewith as a high vacuum distillation to separate mixtures of organic (or silicon) compounds that will not tolerate prolonged heating without excessive structural change or decomposition. It utilizes the heat of condensation as a prime body for radiant heat emission to the surface film of the evaporator. The path between evaporator and condenser is unobstructed. With short residence time and lower distillating temperatures, thermal hazard to the organic material is greatly reduced.

The process of the invention using short path distillation may be effected using commercially available apparatus, e.g. as commercialised by UIC GmbH, D-63755 Alzenau-Hörstein, Germany. A convenient setup is e.g. as illustrated in the Figure.

Short path distillation is preferred. It allows short heating time of the mixture which it is intended to purify, as well as cyclical processing, with corresponding improvement in yield of purified product. Further, thickness of the material on the evaporator wall is reduced, allowing lower evaporation temperature and shorter residence time. Very efficient separation from contaminants is achieved thereby, without need for further purification steps such as by chromatography or recrystallization, or using large amounts of charcoal.

Thus, starting from a raw terbinafine base product containing for example from about 10 ppm to about 200 ppm, e.g. about 50 ppm palladium, and/or from about 10 ppm to about 100 ppm, e.g. about 30 ppm copper, one-step short path distillation results in a product containing less than 1 ppm copper, and/or less than 2 ppm palladium as determined using conventional analytical methods such as atom absorption spectroscopy.

“ppm” herein means parts per million, on a weight by weight basis.

Other, non-metal contaminants, if present, in particular organic compounds, such as (methyl)(naphthalen-1-ylmethyl)amine (by-product 1); 2,2,7,7-tetramethylocta-3,5-diyne (by-product 2); and the Z-isomer of terbinafine, had been believed to be eliminated thereby only partially or not at all, e.g. by-product 1 and the Z-isomer of terbinafine.

However, in-depth investigation and refinement of the methods used for detection of contaminants have now shown that, against expectations, some further, organic contaminant compounds can be eliminated or vastly reduced using the process of the invention.

Such further, non-metal contaminants are e.g. one or more of the following compounds: 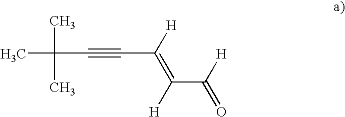
i.e. 6,6-dimethyl-2-hepten-4-ynal; 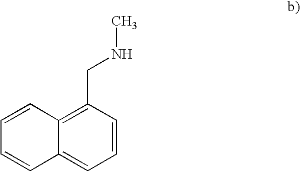
i.e. N-methyl-1-naphthalenemethanamine (by-product 1); 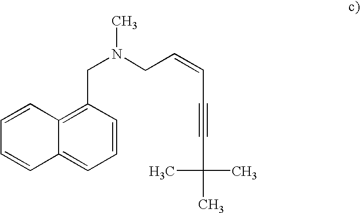
i.e. (Z)-N-(6,6-dimethyl-2-hepten-4-ynyl)-N-methyl-1-naphthalenemethanamine; 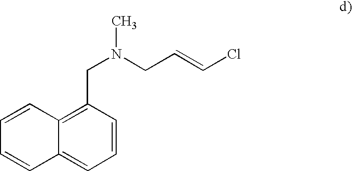
i.e. (E)-N-(3-chloro-2-propenyl)-N-methyl-1-naphthylmethanamine; and, in particular, 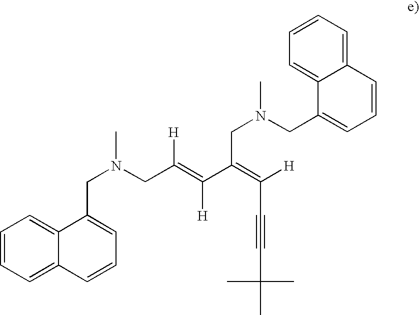
i.e. (E)-4-[4,4-dimethylpentyn-(E)-ylidene]-N¹,N⁵-dimethyl-N¹,N⁵-bisnaphthalen-1-ylmethyl-pent-2-en-1,5-diamine or
2(E),4(Z)-N-(4-[(N′-methyl-N′-1-naphthylmethyl)aminomethyl]-8,8-dimethyl-2,4-nonadien-6-inyl)-N-methyl-1-naphthylmethanamine (substance A).

Detection of non-metal contaminants is preferably effected using an analytical method sensitive to concentrations below the conventional quantitation limit of about 0.05% w/w (500 ppm) usually achieved with conventional detection methods such as reversed-phase high pressure liquid chromatography (RP-HPLC), preferably down to a quantitation limit of around 0.0001% (1 ppm), such as RP-HPLC with UV detection, as achieved with commercially available apparatus such as HP 1100 (Agilent) and Alliance 2695 (Waters) and described in Example 4 hereunder. A typical result is e.g. as illustrated in the Chromatogram hereunder.

Thus starting from a crude terbinafine product comprising e.g. from about 60 to about 80 ppm substance A, a purified product comprising only about 5 ppm substance A may be obtained (see Example 4), while the total amount of other impurities detected is about halved.

Terbinafine has the trans configuration and is usually commercialised in pharmaceutical compositions, such as tablets, where the active ingredient is in the form of the hydrochloride acid addition salt. The free base must therefore be converted to an acid addition salt such as the hydrochloride and it is known from e.g. CH patent No. 678527 that when a crude mixture of terbinafine free base comprises significant amounts of e.g. cis isomer impurity, pure trans isomer may be obtained by effecting salt formation with acid, e.g. hydrochloric acid under simultaneous salt precipitation. Preferably this is effected in the presence of an ester of an organic acid, such as ethyl acetate, or of a mixture of an ester of an organic acid such as ethyl acetate and further organic solvents.

It has now also been found that, surprisingly, such salt formation under simultaneous precipitation of pure trans isomer may advantageously be used together with the above process of the invention for the further removal of contaminants, either metal or non-metal contaminants as described above, resulting in the preparation of exceptionally pure terbinafine in salt form.

Thus starting from a crude product comprising e.g. from about 60 to about 80 ppm substance A, a purified product in salt form may be obtained after distillation and salt formation/precipitation, comprising an amount of substance A below the detection limit, i.e. less than about 1 ppm (see Example 5).

The invention thus further comprises a process for the purification of terbinafine comprising subjecting crude terbinafine in free base form to distillation, additionally together with salt formation of the resultant product under simultaneous precipitation of pure trans isomer, and recovering the resultant product in free base or acid addition salt form.

Salt formation with precipitation is effected in one step. An appropriate solvent is e.g. an ester of an organic acid, or a mixture of an ester of an organic acid and further organic solvents. A preferred ester of an organic acid is e.g. an ester of acetic acid, e.g. a C₁₋₄alkyl ester of acetic acid, such as the methyl, ethyl, n-butyl or isobutyl ester, especially ethyl acetate.

A further organic solvent is e.g. the alcohol corresponding to the ester, e.g. ethanol together with ethyl acetate, isopropanol together with acetic acid isopropyl ester, etc., especially ethanol together with ethyl acetate.

A further organic solvent is e.g. an aliphatic ketone, preferably methyl isobutyl ketone.

The temperature preferably is as conventional, preferably from about −25° to about 100°, preferably about room temperature.

Salt formation with simultaneous precipitation preferably is effected with mineral acids, preferably hydrochloric acid, e.g. as gas or in aqueous solution, e.g. when the solvent is methyl isobutyl ketone, with about 5% to about 40% aqueous hydrochloric acid, at pH 1-3 and at a temperature of from about 10° to about 30°.

The invention thus includes i.a.:
a process for the purification of terbinafine which comprises subjecting crude terbinafine in free base form to distillation and recovering the resultant product in free base or acid addition salt form;
a process of the invention which comprises short path distillation;
a process as defined above wherein distillation is effected at a temperature above 100° C. and under reduced pressure;
a process as defined above wherein the crude terbinafine is prepared using a palladium- and/or a copper-containing catalyst;
a process as defined above wherein the purified product contains less than 2 ppm palladium and/or less than 1 ppm copper;
a process as defined above wherein the crude terbinafine contains more than 2 ppm palladium and/or more than 1 ppm copper, and the resultant purified product contains less than 2 ppm palladium and/or less than 1 ppm copper;
a process as defined above wherein at least 5 kg purified product in free base form is prepared per distillation batch or run, preferably at least 50 kg, especially at least 200 kg;
a process as defined above wherein the crude terbinafine in free base form is prepared by reaction of (E)-N-(3-halo-2-propenyl)-N-methyl-N-(1-naphthylmethyl)amine with 3,3-dimethyl-1-butyne in the presence of a palladium and/or a copper catalyst;
purified terbinafine in free base or acid addition salt form whenever prepared by a process as defined above; and
terbinafine in free base or acid addition salt form comprising less than 2 ppm palladium and/or less than 1 ppm copper, whenever obtained from a crude product in free base form comprising more than 2 ppm palladium and/or more than 1 ppm copper;
   and further:
i) a process as defined above wherein the crude terbinafine comprises non-metal contaminants;
ii) a process as defined above wherein the crude terbinafine comprises more than about 5 ppm of a non-metal contaminant selected from a compound as defined under a), b), c), d) and/or e) above;
iii) a process as defined above wherein the crude terbinafine comprises more than about 5 ppm compound as defined under e) above (i.e. substance A);
iv) a process as defined above wherein the crude terbinafine comprises more than about 5 ppm substance A and the purified terbinafine comprises less than about 5 ppm substance A;
v) a process as defined above comprising additionally salt formation of the resultant product under simultaneous precipitation of pure trans isomer, and recovering the resultant product in free base or acid addition salt form;
vi) a process as defined above including salt formation/precipitation as defined above wherein the crude terbinafine comprises non-metal contaminants; e.g. more than about 5 ppm of a non-metal contaminant selected from a compound as defined under a), b), c), d) and/or e) above; e.g. more than about 5 ppm substance A; e.g. wherein crude terbinafine base comprises more than about 5 ppm substance A and purified terbinafine after distillation and salt formation/precipitation comprises less than about 1 ppm substance A;
vii) use of a process as defined above, optionally additionally together with salt formation of the resultant product under simultaneous precipitation of pure trans isomer and recovery of the resultant product in free base or acid addition salt form, for the preparation of purified terbinafine;
viii) use as defined above for the preparation of purified terbinafine comprising less than about 1 ppm substance A;
ix) purified terbinafine in free base or acid addition salt form containing substance A wherein the concentration of substance A is less than about 1 ppm;
x) purified terbinafine in free base or acid addition salt form whenever prepared by a process as defined under any one of i) to vi) above;
xi) purified terbinafine in free base or acid addition salt form containing less than about 5 ppm substance A, whenever prepared by a process as defined under any one of i) to iv) above; or containing less than about 1 ppm substance A, whenever prepared by a process as defined under v) or vi) above;
xii) a pharmaceutical composition comprising purified terbinafine in free base or acid addition salt form together with one or more pharmaceutically acceptable carrier or diluent, whenever prepared by a process as defined under any one of i) to vi) above;
xiii) a method of producing pure terbinafine, e.g. comprising less than about 1 ppm substance A, wherein the level of substance A present in a crude sample of terbinafine is reduced;
xiv) a method of removing substance A from terbinafine which comprises distilling terbinafine in free base form;
xv) a method of monitoring the level of non-metal contaminant such as substance A in a process as defined under any one of i) to iv) above, comprising removing a sample of crude terbinafine base before distillation and a sample of purified terbinafine base after distillation, and assessing the level of non-metal contaminant such as substance A therein;
xvi) a method of monitoring the level of non-metal contaminant such as substance A in a process as defined under v) or vi) above, comprising removing a sample of crude terbinafine base before distillation, a sample of purified terbinafine base after distillation and a sample of further purified terbinafine after salt formation under precipitation, and assessing the level of non-metal contaminant such as substance A therein.

### EXPLANATION OF THE FIGURE (1/2)

1. Outflow of distillate
2. Connection to vacuum pump
3 Heat inflow
4. Condensor
5. Space under reduced pressure
6. Rolling wipers (distribute crude product evenly to form a film)
7. Heating jacket
8. Sealing liquid, intake
9. Flange for gearing
10. Crude product input
11. Outlet for heat medium
12. Residue outflow
13. Inlet for cooling water
14. Outlet for cooling water

### EXPLANATION OF THE CHROMATOGRAM (2/2)

I=Blank chromatogram (solvent)
II=Reference solution 3 (I ppm substance A)
III=Test solution (pure terbinafine; no substance A detected)
IV=“SST” solution (pure terbinafine, spiked with 5 ppm substance A)
V=Reference solution 2 (100 ppm substance A)
1=Drug substance terbinafine
2=RS substance A
WVL=wavelength 280 nm
abscissa: min (minutes)
ordinate: mAU=absorption units×10⁻³
(see also Example 4)

The following Examples illustrate the invention. All temperatures are in degrees Centigrade. 1000 mbar=750.06 mmHg.

### EXAMPLE 1

### Batch Distillation (Laboratory Scale)

100 g of crude terbinafine base containing 0.3 area-% of (methyl)(naphthalen-1-ylmethyl)amine (by-product 1) are mixed with 20 g peanut oil and the mixture is heated to 1420 at 0.3 mbar pressure (jacket temperature 190°). After 2 hours, 96.4 g of purified terbinafine base as a yellowish distillate and 21.4 g of a dark brown residue are obtained. Due to the thermal impact during batch distillation (2 hours at 142°) the distillate contains about 1 area-% of (methyl)(naphthalen-1-ylmethyl)amine (by-product 1) as determined by gas chromatography (experimental conditions: as for Example 2).

For large scale production the distillation time and the thermal impact would be considerably higher. As a consequence a significantly higher concentration of by-product 1 can be expected unless the distillation time is kept short, such as with e.g. “short path” distillation.

The crude terbinafine base used as a starting material is prepared by reaction of (E)-N-(3-chloro-2-propenyl)-N-methyl-1-naphthalenemethanamine and 3,3-dimethyl-1-butyne in n-butylamine and water in the presence of catalytic amounts of copper(I)iodide and bis-(triphenylphosphine)palladium(II)-dichloride along the lines as described in Example 13 of EP 421302 A2, but without submitting the resultant product to silica gel chromatography.

### EXAMPLE 2

### Short Path Distillation (Laboratory Scale)

In a commercial thin-film evaporator (from Leybold-Heraeus GmbH, Hanau, Germany: diameter of heated drum 7 cm; length 25 cm; cooling finger at 50°; pressure 0.2 mbar; Teflon® rotor at 450 rpm) 179 g crude terbinafine base (prepared as described in Example 1 above) are mixed with 8.9 g peanut oil and the mixture is heated to 50°. After evacuation of the whole system to 0.2 mbar, distillation starts by slowly dropping the mixture into the high temperature zone (jacket temperature 160°) where the terbinafine base is heated to the boiling point for only a few seconds. After 2 hours, 171 g (95%) of purified terbinafine base as a yellowish distillate is obtained, which is contaminated with 1 ppm palladium and less than 1 ppm copper. The chemical purity of the distillate is 98.6% terbinafine base (i.e. E-isomer) as determined by gas chromatography (HP-1 column; crosslinked methyl siloxane; length 30 m; film thickness 2.65 μm; column internal diameter 0.53 mm; flame ionization detector (FID) temperature 300°; injector temperature 250°; temperature gradient 50° to 270°; heating rate 20′/min). In addition 10.5 g of distillation residue and 0.4 g of an oily sublimate were obtained. The sublimate consists mainly of 2,2,7,7-tetramethylocta-3,5-diyne (by-product 2).

The overall purity of terbinafine base as determined by gas chromatography is as follows:

| | Before distillation | After distillation |
|---|---|---|
| | (crude product) | (pure product) |
| By-product 1 (area-%) | 0.1 | 0.1 |
| by-product 2 (area-%) | 0.7 | 0.2 |
| Z-isomer (area-%) | 0.3 | 0.3 |
| E-isomer (weight-%) | 95.6 | 98.6 |
| Pd (ppm) | 177 | 1 |
| Cu (ppm) | 19 | <1 |

### EXAMPLE 3

### Short Path Distillation (Industrial Scale)

Distillation of crude terbinafine base is carried out in a fine vacuum distillation apparatus (UIC GmbH KD 150) using short-path distillation with two serial evaporators. Hereby the material is constantly fed and distributed to the inner surface of a vertically oriented evaporator. As the liquid flows downward, an axially arranged roller wiper system distributes this liquid as a thin film which is constantly mixed (see Figure). This gentle distillation method therefore reduces both the maximum evaporation temperature and the residence time at high temperature.

The starting temperature values are typically set as follows:

internal limit of feeding tank: 70°;
internal limit of product receiver: 80°; jacket limit of residue tank: 80°;
upper and lower internal limits of evaporators 1 and 2: 100°;
jacket limit of evaporators 1 and 2: 160°.

After control of the whole apparatus for emptiness and cleanliness the maximum vacuum of both evaporators which can be reached by the diffusion pumps is checked:

| | |
|---|---|
| before and after evaporator 1: | 1.6 × 10⁻¹ mbar; |
| before evaporator 2: | 2.6 × 10⁻² mbar; |
| after evaporator 2: | 4.7 × 10⁻³ mbar. |

A mixture of 872.5 kg crude terbinafine base (prepared analogously as described in Example 1 above) and 120 kg peanut oil is then transferred to the feeding tank. The peanut oil will ensure that no crusts will build up inside the evaporators. The cooling trap is filled with a mixture of 20 to 30 kg dry ice and about 30 l of ethanol (94%), and temperature values are adjusted as follows:

| | |
|---|---|
| jacket of the residue receiver: | 40°; |
| jacket of evaporator 1: | 120°; |
| condenser of evaporator 1: | 50°; |
| jacket of evaporator 2: | 155°; |
| condenser of evaporator 2: | 45°. |

The internal temperature of the main receiver is set to 50° as the melting point of the product is around 42°.

When all temperatures are reached the crude product is fed to evaporator 1 with a flow of about 1.5 l/min. The distillate (rest of solvents) of evaporator 1 can be collected in the gauge as its volume is small. The residue of evaporator 1 is transferred to evaporator 2 to distill the crude base, which is collected in the heated main receiver (1.4 l/min) as a yellow liquid.

When all the crude mixture is distilled (around 11 h) the residue of evaporator 2 is transferred to the feeding tank and distilled again. Thereby the jacket temperature of evaporator 1 is reduced to 110° and the jacket temperature of evaporator 2 is reduced to 140°.

After the distillation of the residue is completed (around 2 h) the new residue will be cycled through the evaporators until the flow of the product has reached around 0.2 l/h. Before the cycling can be started the jacket temperature of evaporator 1 is reduced to 100° and the condenser temperature of evaporator 2 is increased to 60°. During the cycling the received distillate becomes darker.

At the end of the distillation (overall about 22.5 h) the apparatus is released with nitrogen. The product from the main receiver is filled at around 50° into drums. A sample is taken and the drums are weighed. The chemical purity of the free base is 97% or higher (here it was 98.4%) as determined by gas chromatography. The yield was 856.1 kg. The amount of copper and/or palladium left was very small or undetectable (less than 1 ppm).

The remaining residue (around 120 kg peanut oil; here it was 128 kg), the distillate of evaporator 1 and the condensates of the cooling traps are combined and incinerated. After five to six batches a cleaning of the apparatus is effected.

### COMPARATIVE EXAMPLE

### Charcoal Treatment (Laboratory Scale)

To 404 g of a solution of crude terbinafine base in cyclohexane (prepared analogously as described in Example 1 above from 100 g (E)-N-(3-chloro-2-propenyl)-N-methyl-1-naphthalene-methanamine) is added 10 g activated charcoal (Norit Supra®). The mixture is stirred for 17 hours at 20-25° and then filtered. After evaporation of the solvent at reduced pressure 110.5 g (89%) terbinafine base is obtained, which is contaminated with 14 ppm palladium. The chemical purity of the oily residue is 95% as determined by gas chromatography (experimental conditions: as for Example 2).

### EXAMPLE 4

### Short Path Distillation and RP-HPLC with UV Detection

An industrial amount of 872.5 kg crude terbinafine base from two batches containing 80 ppm (in the other batch: 62 ppm) substance A (as determined from a sample of crude terbinafine base by RP HPLC analysis with UV detection) and 2.45% other detectable non-metal contaminants altogether (in the other batch: 2.40%) is subjected to short path distillation (both batches combined) as described in Example 3 above, and a sample of crude terbinafine base is taken from the distillate and again submitted to RP HPLC analysis. It is found that that sample still contains only 5 ppm substance A and 1.14% other detectable non-metal contaminants altogether.

RP HPLC with UV detection is effected as follows:

Reagents:

Acetonitrile: e.g. LiChrosolv® (Merck);
water: e.g. LiChrosolv® (Merck);
triethylamine: e.g. puriss. p.a. (Fluka);
solvent: acetonitrile or acetonitrile/water 8:2 (v/v);
substance A for comparison (e.g. isolated by silica gel chromatography as a by-product from terbinafine synthesis according to process a) of EP 24587, with spectroscopic confirmation of its chemical structure).
   Apparatus: HP 1100 (Agilent), Alliance 2695 (Waters)
   Column: XTerra RP18, 3.5 μm particle size, length 150 mm, internal diameter 3.0 mm
   Chromatographic Conditions:
Mobile phase:
   A: water/triethylamine 1000:1 (v/v);
   B: acetonitrile/triethylamine 1000:1 (v/v)

gradient:

| Time (min) | Phase A (%) | Phase B (%) |
|---|---|---|
| 0 | 43 | 57 |
| 8 | 33 | 67 |
| 12 | 5 | 95 |
| 13 | 5 | 95 |
| 13.1 | 33 | 57 |
| 16 | 33 | 57 |

next injection

flow rate: 1.0 l/min
detection wavelength: UV absorption at 280 nm
temperature: 52°
injection volume: 20 μl of test and reference solutions
run time: 16 minutes
sample concentration: 40 mg/ml

System suitability is calibrated with reference solutions for:
Repeatibility (reference solution 2, containing 100 ppm substance A, prepared by dilution of 2.0 ml reference solution 1 with solvent to 20.0 ml; reference solution 1, containing 1000 ppm substance A, is obtained by weighing about 2 mg substance A with an accuracy of ±0.001 mg into a 50 ml volumetric flask, dissolving in solvent and diluting therewith to 50 ml);
reporting limit (reference solution 3, containing 1 ppm substance A, prepared by dilution of 2.0 ml of reference solution 2 with solvent to 20.0 ml and dilution of a 2.0 ml aliquot of that solution with solvent to 20.0 ml); and
selectivity (“SST” solution, prepared by weighing about 200 mg test substance with an accuracy of ±0.1 mg into a 5.0 ml volumetric flask, adding 250 μl of reference solution 2 and diluting to volume with solvent: it contains 100% drug substance, spiked with 5 ppm substance A).

Solvent alone is also used for a blank chromatogram. 2 test solutions are prepared by weighing about 200 mg test substance with an accuracy of ±0.1 mg into a 5.0 ml volumetric flask, and dissolution therein, and dilution to volume, with solvent.

It is preferable to use amber glass flasks and vials.

Other non-metal impurities may be detected using similar conditions, e.g. a reversed-phase column Hypersil ODS of 5 μm particle size, with mobile phase A water containing 0.1% triethylamine (v/v), phase B methanol containing 0.1% triethylamine (v/v), solvent methanol or methanol/water 80:20 (v/v) and sample concentration 0.5 mg/ml, at 400 column temperature.

The peak areas for substance A in the chromatogram of the test solution and of reference solution 2 are determined.

Computation is effected as follows (peaks below the reporting limit 1 ppm are disregarded):

$$ppm RS = \frac{{PA}_{T} \times {m}_{R} \times {C}_{R} \times f \times 10000}{{PA}_{R 2} \times {m}_{T}}$$

whereby
RS=terbinafine-related substance, e.g. substance A
PA_{T}=peak area of RS in the test solution
PA_{R2}=peak area of RS in reference solution 2
m_{R}=mass of RS in reference solution 1 (mg)
m_{T}=mass of test substance in the test solution (mg)
C_{R}=content of RS in percent used for reference solutions
f=0.01=dilution factor
10000=conversion factor to ppm

The results from a typical run are as set out in the attached Chromatogram (WVL=wavelength 280 nm; abscissa=minutes; ordinate=mAU=absorption units×10⁻³). The relative retention times for drug substance terbinafine base and substance A are, respectively, 1.00 and about 1.73.

### EXAMPLE 5

### Short Path Distillation Followed by Salt Formation with Precipitation

a) Distillation:

Terbinafine crude base is submitted to short path distillation as described in Example 4 above. The resultant purified terbinafine base comprising 5 ppm substance A as determined by RP HPLC analysis with UV detection is then subjected to salt formation with precipitation:

b) Salt Formation with Precipitation of Trans Isomer:

To the base product from step a) is added ethyl acetate and that mixture is stirred at 20° until full dissolution, the resultant solution is filtered (2 μm) and the pressure reduced to 0.5 bar at 20° temperature. Hydrochloric acid gas is then introduced at 20° to 25°. The pH is maintained at about 2. The resultant suspension is stirred for 4 to 15 hours at 20°, centrifuged, the product obtained is washed with ethyl acetate, centrifuged at 1000 rpm, and the resultant product is dried. Pure terbinafine hydrochloride is obtained. A sample is taken and subjected to RP HPLC analysis. It is found to contain less than 1 ppm substance A.

1. A process for the purification of terbinafine which comprises subjecting crude terbinafine in free base form to distillation and recovering the resultant product in free base or acid addition salt form.

2. A process according to claim 1 which comprises short path distillation.

3. A process according to claim 1 wherein distillation is effected at a temperature above 100° C. and under reduced pressure.

4. A process according to claim 1 wherein the crude terbinafine is prepared using a palladium- and/or a copper-containing catalyst.

5. A process according to claim 4 wherein the purified product contains less than 2 ppm palladium and/or less than 1 ppm copper.

6. A process according to claim 4 wherein the crude terbinafine contains more than 2 ppm palladium and/or more than 1 ppm copper, and the resultant purified product contains less than 2 ppm palladium and/or less than 1 ppm copper.

7. A process according to claim 1 wherein at least 5 kg purified product in free base form is prepared per distillation batch or run, preferably at least 50 kg, especially at least 200 kg.

8. A process according to claim 1 wherein the crude terbinafine in free base form is prepared by reaction of (E)-N-(3-halo-2-propenyl)-N-methyl-N-(1-naphthylmethyl)amine with 3,3-dimethyl-1-butyne in the presence of a palladium and/or a copper catalyst.

9. Purified terbinafine in free base or acid addition salt form whenever prepared by a process according to any one of claim 1.

10. Terbinafine in free base or acid addition salt form comprising less than 2 ppm palladium and/or less than 1 ppm copper, whenever obtained from a crude product in free base form comprising more than 2 ppm palladium and/or more than 1 ppm copper.

11. A process according to claim 1 wherein the crude terbinafine comprises non-metal contaminants.

12. A process according to claim 11 wherein the crude terbinafine comprises more than about 5 ppm of a non-metal contaminant selected from a compound as defined under a), b), c), d) and/or e) (substance A).

13. A process according to claim 12 wherein the crude terbinafine comprises more than about 5 ppm substance A.

14. A process according to claim 13 wherein the crude terbinafine comprises more than about 5 ppm substance A and the purified terbinafine comprises less than about 5 ppm substance A.

15. A process according to claim 11 comprising additionally salt formation of the resultant product under simultaneous precipitation of pure trans isomer, and recovering the resultant product in free base or acid addition salt form.

16. A process according to claim 15 wherein the crude terbinafine comprises more than about 5 ppm substance A and purified terbinafine after distillation and salt formation/precipitation comprises less than about 1 ppm substance A.

17. Use of a process according to claim 11 for the preparation of purified terbinafine.

18. Use according to claim 17 for the preparation of purified terbinafine comprising less than about 1 ppm substance A.

19. Purified terbinafine in free base or acid addition salt form containing substance A wherein the concentration of substance A is less than about 1 ppm.

20. Purified terbinafine in free base or acid addition salt form whenever prepared by a process according to claim 11.

21. Purified terbinafine in free base or acid addition salt form containing less than about 5 ppm substance A, whenever prepared by a process according to claim 11.

22. A pharmaceutical composition comprising purified terbinafine in free base or acid addition salt form together with one or more pharmaceutically acceptable carrier or diluent, whenever prepared by a process according to claim 11.

23. A method of producing pure terbinafine comprising less than about 1 ppm substance A, wherein the level of substance A present in a crude sample of terbinafine is reduced.

24. A method of removing substance A from terbinafine which comprises distilling terbinafine in free base form.

25. A method of monitoring the level of non-metal contaminant such as substance A in a process according to claim 11, comprising removing a sample of crude terbinafine base before distillation and a sample of purified terbinafine base after distillation, and assessing the level of non-metal contaminant such as substance A therein.

26. A method of monitoring the level of non-metal contaminant such as substance A in a process according to claim 15, comprising removing a sample of crude terbinafine base before distillation, a sample of purified terbinafine base after distillation and a sample of further purified terbinafine after salt formation under precipitation, and assessing the level of non-metal contaminant such as substance A therein.

27. Purified terbinafine in free base or acid addition salt form containing less than about 1 ppm substance A, whenever prepared by a process according to claim 15.
